(19) United States
(12) Patent Application Publication
(10) Pub. No.: US 2017/0108414 A1
(43) Pub. Date: Apr. 20, 2017

(54) HIGH-RESOLUTION THREE-DIMENSIONAL IMAGING OF MAMMALIAN HEARTS

(71) Applicant: UNIVERSITY OF WASHINGTON, SEATTLE, WA (US)

(72) Inventors: W. Robb MACLELLAN, SEATTLE, WA (US); Danny EL-NACHEF, SEATTLE, WA (US)

(73) Assignee: UNIVERSITY OF WASHINGTON, SEATTLE, WA (US)

(21) Appl. No.: 15/298,960

(22) Filed: Oct. 20, 2016

Related U.S. Application Data
(60) Provisional application No. 62/244,105, filed on Oct. 20, 2015.

(51) Int. Cl. G01N 1/30; G06T 3/40; G01N 33/483; G06T 7/00; G01N 1/36; G01N 1/28

(52) U.S. Cl. CPC G01N 1/30; G01N 1/36; G01N 1/286; G01N 33/4833; G06T 7/0012; G06T 3/4053; G01N 2001/305; G06T 2207/30004

(57) ABSTRACT

Methods and materials for high-resolution three-dimensional imaging of mammalian tissues, such as heart and intestine, are described. Both methods of tissue preparation for imaging and methods of imaging are described, as well as kits comprising materials and media for use in the methods.

This application claims benefit of U.S. provisional patent application No. 62/244,105, filed Oct. 20, 2015, the entire contents of which are incorporated by reference into this application.

### TECHNICAL FIELD OF THE INVENTION

The present invention relates to methods and materials for high-resolution three-dimensional imaging of mammalian tissues, such as heart and intestine. Both methods of tissue preparation for imaging and methods of imaging are descrbed, as well as kits comprising materials and media for use in the methods.

### BACKGROUND OF THE INVENTION

Microscopy has long been appreciated as a methodology to directly study cells. However, the limitations of traditional imaging techniques restrict the type of information that can be gained. To be compatible with high resolution imaging, hearts are either dissociated into single cells or are sectioned into thin slices; resulting in specimens that are eventually mounted on glass slides for viewing. Each method (dissociating vs. sectioning the heart) has pros and cons. Dissociating the heart into single cells requires the use of proteases that degrade the extraceflular matrix that holds cells together, releasing the individual cells. However, these proteases can also damage the cells, and this processing increases the time from living tissue to preserved specimen, meaning the final specimen may not well reflect what occurs in vivo. Though this technique is well suited for studying single cells in detail, information regarding the cells' context in the organ is lost. Tissue sections can offer a view of the cell in situ, but have their own limitations.

Currently, the two most common methods for tissue sectioning in cardiac studies are microtome and cryostat sectioning. The techniques utilize distinct sample preparation. Prior to microtome sectioning, samples are typically formalin-fixed and embedded in paraffin wax (1). Paraffin sections are typically 4-10 microns (μm) thick. The ability of paraffinlmicrotome processing to generate such thin sections was initially thought to be an advantage, as thicker tissue codd not be examined, as light is absorbed/scattered as it passes through thick sections, making the interior of the sections unable to view, even with microscopy techniques that can overcome the issue of out-of-focus signal (e.g., confocal microscopy). Although paraffin sections up to 100 μm thick have been reported in non-cardiac tissues, subsequent imaging was irylited to low-resolution 2D imaging (2), and such thick paraffin sections have not been reported, to our knowledge, for imaging heart tissue. The use of formaldehyde-related fixatives and the paraffin embedding that occurs prior to sectioning requires the resulting sections to undergo harsh processing to expose antigens masked by crosslinking fixatives and remove the paraffin and rehydrate the sample. Cryosectioning frequently utilizes fresh tissue that is cryopreserved, with the resulting sections typically being post-fixed with alcohol and/or acetone, though samples may also be fixed with formalin prior to cryopreservation (3), Cryosections are generally thin (>10 μm); thicker sections can be obtained but section quality is often compromised (rolling, ruffling, or cracking). A major limitation of using frozen sections is that the cryopreservation process can disrupt the morphology of the cells, creating artefacts when imaging. Thus, the two most common sectioning methods are poorly suited for generating thick sections and the processing required for these methods can reduce image quality.

Having thick sections is particuiarly important in the field of cardiology, as mature mammalian cardiac myocytes are hundreds of times larger than many other mamiTialian cell types, frequently larger than 200 μm long and 20 μm wide (4). Thin microtome and cryostat sections cannot capture an individual cardiac myocyte in its entirety. In contrast to these methods, vibratome sectioning is designed to section tissue into 100-1000 μm slices. Intriguingly this method has been used to culture thick sections of living heart tissue, though downstream imaging was performed on thin (10 μm) cryosections that were cut/re-sectioned from the initial 300 μm vibratome section (5).

Generating thick sections presents another hurdle that prevents high-resolution 3D imaging of cardiac tissue. Thick sections present a challenge when imaging opaque tissues, such as the heart and almost all other tissues, with the exception of the retina which is transparent. Reflection, absorption, and scattering of light results in the image appearing blurry from out-of-focus signal, which is more abundant in thick sections. Though the issue of out-of-focus signal can be overcome with conventional confocal microscopy, opaque tissues also prevent iight from reaching and escaping the interior of the section, which precludes fluorescence imaging beyond a few microns of the section's surface, There are methods to make opaque tissue more transparent, a process called tissue clearing. This has been used in embryonic heart studies in model organisms, where the hearts are small enough to avoid the need for sectioning, and can be stained and imaged “whole mount” (6, 7). However, whole mount imaging is not feasible in adult rodent or human sarriples for use with confocal microscopy, or with subcellular resolution in general; technologies that are able to image larger whole mount hearts, such as digital volumetric imaging, have resolution limits of >10 μm. Thus, there remains a need for a sample preparation method that is suitable for high resolution (0.3 μm or better) 3D imaging of mammalian hearts.

### SUMMARY OF THE INVENTION

The invention provides a method of preparing a tissue sample for high-resolution three-dimensional tissue imaging. In one embodiment, the method comprises embedding a fixed tissue sample in an embedding medium; sectioning the embedded tissue into sections of 20-1000 μm thickness; staining the sectioned tissue; mounting the stained 20-1000 μm thick tissue sections on a coverslip and glass slide: and clearing the 20-1000 μm thick tissue sections with a mixture of Benzyl Alcohol and Benzyl Benzoate (BABB). In one embodiment, the staining comprises incubating the sectioned tissue in suspension with fluorescent, luminescent, and/or staining reagents. In one embodiment, the tissue is adult mammalian tissue. In one embodiment, the tissue is cardiac tissue, In another embodiment, the tissue is intestinal tissue.

In some embodiments, the tissue sections are 100-800 μm thick, or 200-700 μm thick, In other embodiments, the sections are 50-500, 50-400, 100-350, 100-400, 100-500, 300-500 μm thick. In some embodiments, the tissue is fixed with formaldehyde, paraformaldehyde, glutaraldehyde, acetone, methanol, ethanol, or isopropanol. In one embodiment, the tissue is fixed in 4% paraformaldehyde in PBS, precooled to 4° C., and incubated about 16 hours at 4° C. In another embodiment, the tissue is fixed in methanol (MeOH) and is rehydrated in a stepwise manner. For example, the tissue can be fixed in 100% MeOH, precooled to −20°, incubated at −20° C. for 30 min to 1 hr: and in a stepwise manner, incubated in precooled 80% MeoH/20% PBS, 60% MeOH/40% PBS, wherein each incubation is about 30 minutes at −20° C. Those skilled in the art appreciate that minor variations in these ratios, times and temperatures can be tolerated, such that the tissue can be incubated at about −20° C. for 30 min to 1 hr; and in a stepwise manner, incubated in precooled MeOH at about 80% MeOH/20% PBS, then about 60% MeOH/40% PBS, wherein each incubation is about 30 minutes at about −20° C. In some embodiments the embedding medium is agarose, histogel, paraffin, or optimal cutting temperature (OCT) compound.

Also provided is a method of high-resolution three-dimensional tissue imaging, In one embodiment, the method comprises preparing a tissue sample according to the method described above, and acquiring images of the tissue with desired resolution, In one embodiment, the desired resolution is about 0.2 μm in xy (lateral resolution), and about 0.3 μm in yz and xz (axial resolution). In some embodiments, the images are obtained with a 60× or 100× objective. Though not making full use of the innovations ability to achieve very high resolution, lower power objectives can be used. In some embodiments, the acquisition of images comprises one or more of the following imaging methods: single or multi-photon confocal, spinning disc, light sheet, stochastic optical reconstruction microscopy (STORM), photoactivated localization microscopy (PALM), stimulated emission depleted (STED), near-field scanning optical microscopy (NSOM), Structured Illumination (SIM), or super-resolution optical fluctuation imaging (SOFI). In some embodiments, the staining comprises use of a fluorescent or luminescent protein.

The invention additionally provides a kit comprising items and solutions for use in carrying out the methods described here. In one embodiment, the kit comprises a plurality of pre-coated adherent ultra-thin-coverslips (#0). BABB, embedding medium, a plurality of baskets comprising 100 μm or smaller mesh for in-suspension staining in wells, and at least one container. Optionally, the kit further comprises a package insert containing instructions for use. Instructions Can be provided on printed or electronic media. Also provided is a tissue processing system comprising the materials and services described herein for embedding, sectioning, staining, mounting, and imaging tissue.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Conventional cardiac histological techniques. (A) Paraffin section of adult mouse heart stained with cardiac myocyte marker alpha actinin (green) and Hoechst DNA stain (blue), 20× objective. (B) Cryostat section of postnatal heart stained with cardiac myocyte marker alpha actinin (green) and Hoechst DNA stain (blue, grayscale image below), 60× objective.

FIG. 2: Thick cardiac slides using vibratome sectioning combined with tissue clearing techniques. (A) Pictures of prepared slides with different preparation methods of 100 μm thick sections. Fixed sections appear opaque with standard mounting protocol, however, after clearing and mounting with BABB, sections are nearly completely transparent. (B) When looking at the surface of the section in the XY view, both non-cleared and cleared images appear reasonably stained, however, when looking at the 30 μm of depth in the XZ or YZ views, WGA signal is lost within a few microns of the surface in non-cleared samples, while in BABB cleared samples, signal is retained throughout the depth. 20× objective.

FIG. 3: Example images from serial 2D image series spanning 20 μm of thickness of mouse intestine. Mouse intestine was processed using our protocol for hearts, though WGA staining shows tissue, and some cell outlines can be discerned, overall tissue was not cleanly sectioned with this protocol.

FIG. 4: Various views of a 3D reconstruction from methanol-fixed adult mouse heart stained with phalloidin (red), WGA (green), and Hoechst (blue). Volume represents 26.4 μm of thickness, 0.3 μm z-steps.

FIG. 5: Example images from serial 2D image series that was used to generate 3D reconstruction in FIG. 4.

FIG. 6: PFA-fixed adult mouse hearts. Example images from serial 2D image series spanning 24 μm. Phalloidin (red), WGA (green), and Hoechst (blue).

FIG. 7: imaging 100 μm depths. Top. Example images from serial 2D image series spanning 100 μm of thickness with 5 μm z-step, Phalloidin (red), WGA (green), and Hoechst (blue), Bottom, orthogonal view of 3D reconstructed image. Note cardiac myocytes cut transversally can be see along their long axis in xz and yz.

FIG. 8: Various views of 3D reconstruction of adult mouse heart stained with phalloidin (red), WGA (green), and Hoechst (blue, or grayscale). Volume represents 36.3 μm of thickness, 0.3 μm z resolution. Note that DNA structure can be visualized in the nuclei (Hoechst stain).

FIG. 9: 3D rendering of individual nuclei that were cropped from the images in FIG. 8. Images show nuclei over 180 degrees of rotation along the y-axis. Note the intensely staining heterochromatin, or densely packed DNA, structures within the nuclei visible with Hoechst staining.

FIG. 10: Immunostaining markers of heterochromatin in adult mouse hearts. (A) 3D reconstruction of 60 μm thickness, 1 μm z-steps. Histone H3K9 tri-methylation (red) and Heterochromatin Protein 1 gamma (green) co-localize, resulting in heterochromatin on the nuclear periphery and internal foci appearing yellow, (B) Magnification of A. (C) Example of nuclei within 2D images used to generate A and B, Hoechst (blue) is shown, (D) 3D reconstruction where Chromatin is clearly visualized, Hoechst (red) and H3K9m3 (green), while larger features, such as arteriole branching (WGA, grayscale), are also apparent. 14.1 μm volume, 0.3 μm z-steps.

FIG. 11: Example images from serial 2D image series spanning 41 μm of thickness in human ischemic heart disease patient samples, Phalloidin (red), WGA (green), and Hoechst (grayscale). Note the perivascular and interstitial fibrosis.

FIG. 12: Orthogonal views of 3D reconstructed image generated from the serial 2D image series from FIG. 11, 1 μm z-steps. The yz-axis is being pushed toward the back of the image in this sequence of images, showing transverse views of cardiac myocytes that were sectioned in the longitudinal orientation.

FIG. 13: Example images from serial 2D image series spanning 58 μm of thickness in human ischemic heart disease patient samples. Phalloidin (red), WGA (green), and Hoechst (blue), Sarcomere disarray and fibrotic areas are clearly visualized.

FIG. 14: Non-cardiac mycote nucleus is easily confused as a cardiac myocyte nucleus with 2D imaging. (A) Desmin immunostaining (red) stains cardiac myocyte sarcomere, and Hoechst (blue) stains DNA. (B) Non-cardiac myocytes are easily identified with 3D context and the addition of plasma membrane marker WGA (green).

FIG. 15: Example images from serial 2D image series spanning 63 μm of thickness in normal mouse hearts after TAC surgeries. Mitotic marker, pH3 (grayscale) was immunostained along with Phalloidin (red), WGA (green), and Hoechst (blue). Two mitotic non-cardiac myocytes can be seen in frames 5 and 8, though no cardiac myocytes stained positive for pH3.

FIG. 16: Various views of a 3D reconstruction from the 2D image series shown in FIG. 15, 1 μm z-steps. Note dear visualization of cardiac myocyte cross section in the yz view. Sarcomeres and nuclei are dearly visibly in the xy, xz, and yz views.

FIG. 17: Various orthogonal views of the 3D reconstruction shown in FIG. 16, 1μm z-steps. Note clear visualization of cardiac myocyte cross section in the yz view. Sarcomeres are clearly visibly in the xy, xz, and yz views. Also, a pH3-positive, non-cardiac myocyte is seen.

FIG. 18: Example images from serial 2D image series spanning 63 μm of thickness in transgenic mouse hearts after TAC surgeries. Mitotic marker, pH3 (grayscale) was immunostained along with Phalloidin (red), WGA (green), and Hoechst (blue) Several cardiac myocytes were positive for pH3, indicating mitotic activity.

FIG. 19: Orthogonal view of 3D reconstruction of images from FIG. 18, 1 μm z-steps. pH3 positive nuclei are unambiguously assigned as cardiac myocyte or non-cardiac myocyte by viewing nuclei position within cell in 3D.

FIG. 20: Example of volume quantification of DNA domains within adult mouse cardiac myocyte nucleL Hoechst DNA stain (grayscale) is segmented in 3D. The resulting masks are shown (magenta). Multiple 2D images within the 3D dataset are shown as single-channel or merged images. Displayed are the 5 heterochromatin/DNA foci in this nucleus. The volume of each foci is as follows: 1=2.344 μm³, 2=2.67 μm³, 3=1.404 μm³, 4=0.273 μm³, and 5=0.592 μm³.

FIG. 21: Simultaneous staining of cell boundaries, blood vessels, and markers of heterochromatin in adult mouse hearts, 3D reconstruction of 60 μm thickness, 1 μm z-steps. Histone H3K9 tri-methylation (red) DNA (blue) and cell membranes (green). (A) Blended 3D projection showing the XY and XZ surfaces are uniformly labeled through the depth of several cardiac myocytes, demonstrating the effectiveness of the tissue clearing and staining protocol. Blue arrow points to large vessel lumen (B) Maximum intensity 3D projection, blue arrows point to a single large vessel that spans the section, starting from the superficial bottom right, to the deep top left. (C) The lumen of the large vessel is seen as an absence of signal that spans through the entire section, allowing quantification of large blood vessel volumes while simultaneously having resolution capable of quantifying the volumes of subnuclear domains (FIGS. 9, 10 and 20).

FIG. 22. Apoptosis detection throughout thick sections, (A) TUNEL staining in untreated (left) and DNasel treated (right) samples demonstrating DNasel treatment results in TUNEL+ nuclei, as expected. (B) 3D reconstruction of TUNEL staining in DNasel treated samples.

### DETAILED DESCRIPTION OF THE INVENTION

The invention provides new tools to understand the molecular and cellular processes that govern mammalian heart physiology and pathophysiology, which are fundamental to the goal of better treating and diagnosing diseases of the heart. We herein describe a sample preparation methodology that allows high-resolution 3D-imaging in thick tissue sections that originate from large specimens, such as adult mammalian heart. The methods described herein make it possible to generate sections that are thick enough to retain anatomical features of adult mammalian organs with many layers of cells in their native context, yet thin enough to be compatible with the limited working distance of high resolution objectives, staining, and making these thick sections transparent and mounted in a manner that is viewable under a high resolution objective to generate high resolution three-dimensional images. This ability to prepare tissue sections thick enough to permit sufficient inclusion of cellular structure while also dear enough for imaging is an unexpected and needed improvement over prior methods and the faded attempts of others.

We compare the method with traditional techniques using mouse tissue. In human ischemic heart disease samples, we clearly visualize sarcomere disarray, with resolution of single sarcomeres, myocyte DNA content, and fibrosis all in the context of entire cells within native tissue. In a mouse model of cardiac myocyte hyperplasia, we demonstrate increased mitotic activity using this methodology and directly quantify the proliferating myocytes per volume. We examine apoptosis and perform additional immunofluorescence assays to determine chromatin subnuclear conformation. In these assays, resolutions of 0.3 microns or better are achieved for all three dimensions, throughout the entire depth of 100 micron-thick sections, for the first time allowing high resolution imaging in sections that span several cardiac myocytes in thickness.

### Definitions

All scientific and technical terms used in this application have meanings commonly used in the art unless otherwise specified. As used in this application, the following words or phrases have the meanings specified

As used herein, “high-resoution” means a resolution of at least 10 μm. In some embodiments, the resolution is 0.3 μm or better in all three dimensions (XY, YZ, XZ).

As used herein, “protein-binding reagents” means protein, peptide, or other chemical moiety that binds to a protein. Some protein-binding reagents, such as antibodies and antibody fragments, specifically bind a target protein.

As used herein, “staining” refers to application or administration of a detectable label that enables visualization of tissue structures via microscopy. The staining methods can be conventional histological stains, immunohistochemical and other binding reagent based methods, and/or labeling that occurs via genetically encoded proteins (such as GFP).

As used herein, “clearing” a tissue specimen or block means exposing the tissue, typically by immersion, to a substance that imparts optical clarity or transparency to the tissue. Typically, a clearing agent is fully miscible in both ethanol and paraffin wax, allowing it to be used after a dehydration step and prior to infiltration with a histological wax.

As used herein, “embedding medium” refers to a medium that changes from liquid to a solid or gel state, allowing embedding of tissue and providing structural support to tissue during tissue sectioning. Examples of embedding media include, but are not limited to, agarose, histogel, paraffin, and optimal cutting temperature (OCT) compound.

As used herein, “a” or “an” means at least one, unless clearly indicated otherwise.

### Overview of Method for High-Resolution Imaging

In the Examples below, we describe the establishment of a sample preparation method that utilizes vibratome sectioning, tissue clearing, and confocal microscopy techniques. This method successfully generated 3D images of adult mammalian hearts with 0.2 μm resolution in xy and 0.3 μm resolution in yz/xz, spanning several cardiac myocytes of thickness, in native tissue. This innovation makes it possible to assess fibrosis and sarcomere disarray in human ischemic heart disease samples, and to obtain volumetric quantification of mitotic activity, apoptosis, and chromatin subnuclear structure in adult mouse cardiac myocytes. The following lists an overview of steps for preparing and imaging tissue.

1. Obtain Tissue Sample

Obtain fresh tissue sample via dissection or extraction. Those skilled in the art understand that the tissue can be obtained from another party (e.g., surgeon, technician) who has performed the dissection or extraction.

2. Fix Tissue

Fix tissue with crosslinking reagent (e.g., formaldehyde, paraformaldehyde, glutaraldehyde) or dehydration reagent (e.g., methanol, ethanol, isopropanol).

3. Embed Tissue

Embed tissue in a medium that can change from liquid to gel/solid phase. Examples include agarose and histogel (liquid at warm temperature and gel at room temperature), waxes (e.g., paraffin) and other mediums, such as OCT (liquid at room temperature, solid at cold temperatures). Utilize these mediums in a manner that the tissue is engulfed in the liquid-phase of the medium, and then switched to gel/solid phase, When gelled or solidified, the medium should have sufficient rigidity to hold tissue in place and provide additional structural support to the tissue to prevent damage to tissue during sectioning.

4. Section Tissue

The tissue is sectioned into thick sections (50-1000 micron thickness). Various sectioning devices can be utilized (e.g., vibratome, microtome, and cryostat modalities). Vibratome is the most suitable for sectioning thick sections, though other sectioning devices can also generate sections of 100 micron or greater thickness. A vibratome may also cut sections thinner than 50 microns and greater than 1000 microns. However, high resolution objectives (e.g., 60× and 100× objectives) cannot focus beyond tissue thickness over 300 microns, Nonetheless, 1000 micron thickness can be generated to view under lower power objectives and objectives with greater working distances.

5. Stain the Entire Thickness of the Thick Sections

The staining uses a protocol that maximizes penetration of antibodies and staining reagents. Fluorescent or luminescent staining reagents (e.g., antibodies, proteins, chemicals) should be incubated for a duration that allows the penetration of the reagents into the deep interior of the thick sections. Depending on the reagent's diffusion rate, incubations can take from 5 minutes to 3 days, Staining the section in suspension with agitation (e.g., rocking) greatly increases penetration of staining reagent and decreases incubation time. Staining in suspension is particularly useful to adequate penetration of dye or antibodies.

6. Mount and Clear Sections

The mounting and clearing is performed in a manner that maximizes viewable volume of sample within limited objective working distance. Samples are preferably adhered or mounted to the coverslip (rather than to the slide) to fully utilize the working distance of the objective lens. To make the tissue transparent, the tissue is placed in a transparency reagent that 1) solubilizes light absorbing molecules (such as lipids), and 2) changes the diffraction index of the tissue to closely match that of glass/immersion oil. Benzyl Alcohol/Benzyl Benzoate mixture (BABB), and other clearing reagents are typically immiscible with the aqueous molecules in tissue and thus stepwise dehydration into alcohols can be done prior to clearing reagent addition, allowing the clearing reagent to enter the tissue.

7. Image

Sections can be viewed under any scope, however, to achieve high resolution 3D imaging, an imaging modality that captures only light that is in the plane of focus (e,g., confocal, spinning disc, light sheet microscopy modalities) is used, Super-resolution imaging methodologies (e.g., STORM, PALM, STED, NSOM, SOFI , etc) can also be used with this methodology to obtain 3D images of thick sections with resolution greater than the light diffraction limit.

### Methods of the Invention

The invention provides a method of preparing a tissue sample for high-resolution three-dimensional tissue imaging. In one embodiment, the method comprises embedding a fixed tissue sample in an embedding medium; sectioning the embedded tissue into sections of 20-1000 μm thickness; staining the sectioned tissue; mounting the stained 20-1000 μm thick tissue sections on a coverslip and glass slide; and clearing the 20-1000 μm thick tissue sections with a mixture of Benzyl Alcohol and Benzyl Benzoate (BABB). In one embodiment, the staining comprises incubating the sectioned tissue in suspension with fluorescent, luminescent, and/or other staining reagents, In one embodiment, the tissue is adult mammalian tissue. In one embodiment, the tissue is cardiac tissue. In another embodiment, the tissue is intestinal tissue.

In some embodiments, the tissue sections are 100-800 μm thick, or 200-700 μm thick. In other embodiments, the sections are 50-500, 50-400, 100-350, 100-400, 100-500, 300-500 μm thick, Low-power objective lenses that are able to view sections thicker than 400 μm (because they have enough working distance, such as a 10× or lower power objective), have especially poor resolution in the z dimension, making them much less useful for 3D reconstructions. There are non-microscopy based methods that are suitable for generating these low resolution 3D images (such as Optical coherence tomography). However, since confocal microscopy is more widely used and available, there is potential utility of 1000 μm thick sections for low power objectives. A particular practical utility of the methods described herein is for sections 100-400 μm, however, to generate high resolution 3D images that are one or two magnitudes of order thicker than traditional methods,

In some embodiments, the tissue is fixed with formaldehyde, paraformaldehyde, glutaraldehyde, acetone, methanol, ethanol, or isopropanol. In one embodiment, the tissue is fixed in 4% paraformaldehyde in PBS, precooled to 4° C., and incubated about 16 hours at 4° C. In another embodiment, the tissue is fixed in methanol (MeOH) and is rehydrated in a stepwise manner. For example, the tissue can be fixed in 100% MeOH, precooled to −20° C., incubated at 20° C. for 30 min to 1 hr; and in a stepwise manner, incubated in precooled 80% MeOH/20% PBS, 60% MeOH/40% PBS, wherein each incubation is about 30 minutes at −20° C. Those skilled in the art appreciate that minor variations in these ratios, times and temperatures can be tolerated, such that the tissue can be incubated at about −20° C. for 30 min to 1 hr; and in a stepwise manner, incubated in precooled Me0H at about 80% MeOH/20% PBS, then about 60% MeOH/40% PBS, wherein each incubation is about 30 minutes at about −20° C. In some embodiments, the embedding medium is agarose, histogel, paraffin, or optimal cutting temperature (OCT) compound.

The staining can involve application or administration of a detectable label that enables visualization of tissue structures via microscopy. Examples of detectable labels include, but are not limited to, conventional histological stains (including protein-, carbohydrate-, lipid-, ion-, and other molecule-binding staining reagents), as well as fluorescent and/or luminescent proteins that can be genetically encoded. One representative example of such a protein is green fluorescent protein (GFP).

Also provided is a method of high-resolution three-dimensional tissue imaging. In one embodiment, the method comprises preparing a tissue sample according to the method described above, and acquiring images of the tissue with desired resolution. In one embodiment, the desired resolution is about 0.2 μm in xy (lateral resolution), and about 0.3 μm in yz and xz (axial resolution). In some embodiments, the images are obtained with a 60× or 100× objective. Though not making full use of the innovations ability to achieve very high resolution, lower power objectives can be used. In some embodiments, the acquisition of images comprises one or more of the following imaging methods: single or multi-photon confocal, spinning disc, light sheet, stochastic optical reconstruction microscopy (STORM), photoactivated localization microscopy (PALM), stimulated emission depleted (STED), near-field scanning optical microscopy (NSOM), Structured Illumination (SIM), or super-resolution optical fluctuation imaging (SOFI). In some embodiments, the staining comprises use of a fluorescent or luminescent protein.

The XY pixel size in images created in one exemplary embodiment of the invention, is 90 nm. With increasing objective power, the most dramatic increases in resolution are observed in the XZ/YZ planes, which is critical when generating 3D images. The methods of the invention have been used to achieve a very high XZ/YZ resolution or voxel size of less than 0.02 microns³, which cannot be achieved with lower power objectives.

Also provided are methods of assessing fibrosis and/or sarcomere disarray in ischemic heart tissue, as well as methods of quantifying mitotic activity in cardiac and other thick tissue sections. The methods comprise imaging the tissue as described herein, and performing the assessment and/or quantification on the imaged tissue.

### Kits & Systems

The invention provides kits comprising materials and solutions for use in carrying out the methods described here. In one embodiment, the kit comprises a plurality of pre-coated adherent ultra-thin-coverslips (#0). BABB, embedding medium, a plurality of baskets comprising 100 μm or smaller mesh for in-suspension staining in wells, and at least one container. Optionally, the kit further comprises a package insert containing instructions for use. Instructions can be provided on printed or electronic media.

In one embodiment, a composition or kit of the invention further comprises one or more, or each of the reagents described in the following examples. Also provided is a tissue processing system comprising the materials and services described herein for embedding, sectioning, staining, mounting, and/or imaging tissue, including an operational protocol to guide service providers in performing such tissue processing.

### EXAMPLES

The following examples are presented to illustrate the present invention and to assist one of ordinary skill in making and using the same. The examples are not intended in any way to otherwise limit the scope of the invention.

### Example 1: High-Resolution Three-Dimensional Cardiac Imaging

In this work we established a sample preparation method that utilizes vibratorne sectioning, tissue clearing, and confocal microscopy techniques. We generated 3D images of adult mammalian hearts with 0.2 μm resolution in xy and 0.3 μm resolution in yz/xz, spanning several cardiac myocytes of thickness, in native tissue. We used this innovation to assess fibrosis and sarcomere disarray in human ischemic heart disease samples and to obtain volumetric quantification of mitotic activity, apoptosis, and chromatin subnuclear structure in adult mouse cardiac myocytes,

We followed established conventional protocols for imaging paraffin sections and cryosections, Hearts for paraffin sections were fixed with 4% PFA and sectioned at 4 μm thiCkneSS. Fresh hearts were frozen in OCT medium, cryosectioned at 10 μm thickness, and fixed with 100% methanol prior to staining, For High-Resolution Three-Dimensional Cardiac Imaging, we tested various fixation, embedding, clearing, mounting, and staining methods on multiple tissues. The optimal procedures used to generate the data in the results section are described in the following examples.

### Example 2: Human Heart Preparation

Obtain human tissue. We used IRB approved LVAD core from patient with ischemic heart disease, keeping fresh sample in a cold, high potassium buffer to keep the myocytes arrested, such as KB buffer.

Transfer tissue to glass plate, with scalpel, trim heart piece into a cube, ˜3 mm×3 mm×3 mm, keeping sample on ice. *Note, larger pieces do not section well.

Fix tissue (Choose fix appropriate for downstream applications, other fixatives may be used):

Transferring sample to 4% paraformaldehyde in PBS, precooled to 4 C. Incubate overnight at 4 C.

### OR

100% methanol (MeOH), precooled to −20 C. Incubate at −20 C for 30 min to 1 hr. In a stepwise manner, incubate the sample in precooled 80% MeOH/20% PBS, 60% MeOH/40% PBS, with each incubation for 30 minutes at −20 C.

Wash 3× with PBS, keep in PBS at 4 C until embedding.

### Example 3: Mouse Heart Preparation

Euthanize animal as approved on animal protocol, according to government and insfitute guidelines, we used isoflurane overdose. Collect heart in cold KB buffer.

(Optional, but recommended) Cannulate aorta with blunt 26 g needle, secure with suture:

Perfuse 4 ml KB buffer, precooled at 4 C, 2 mL/min to wash the blood out of the coronary vessels.

Perfuse Fixative:

4 mL 4% PFA, 4 C, 2 mL/min

### OR

2 mL 100% MeOH, −20 C, 1 mL/min

Fix Tissue:

Transfer sample to 4% paraformaldehyde in PBS, precooled to 4 C. Incubate 16 hrs at 4 C.

100% methanol (MeOH), precooled to −20 C. Incubate at −20 C for 30 min to 1 hr. In a stepwise manner, incubate the sample in precooled 80% MeOH/120% PBS, 60% MeOH/40% PBS, with each incubation for 30 minutes at −20 C.

Wash 3' with PBS, keep in PBS at 4 C until embedding.

### Example 4: Tissue Embedding

Prepare 6% low-melt agarose in PBS, dissolve agarose by microwaving or boiling sample. Let solution cool to 50 C in a water bath incubator. (Histogel and other gelling agents may be used in place of agarose)

Dab the fixed tissue on a Kimwipe™ wiper or gauze and place in a mold, fill with molten agarose. Work quickly, the agarose solidifies at room temperature.

For whole hearts, trim the basal or apical end, fill ventricular chambers with molten agarose using a pipettor, then proceed to fill mold with agarose.

Keep samples at 4 C until sectioning.

We have tried using unembedded tissue, directly mounting the tissue on the vibratome sectioning platform; the agarose, or other medium, embedding is needed to give the sample rigidity during sectioning. The agarose can be left attached in the resulting tissue sections because it is inert and does not interfere with subsequent staining; if desired the agarose can be gently peeled off the tissue section with a brush.

### Example 5: Vibratome Sectioning

Remove agarose-heart block from mold, trim to ˜5 mm×5 mm×5 mm, keeping sample on ice.

Superglue agarose to vibratome sectioning platform. Fill chamber with PBS, precooled to 4° C.

Set section thickness to 100 μm or greater, collect sections with a soft, synthetic-bristle paintbrush and transfer to a 24-well plate, or other vessel, containing PBS.

We have found very slow blade advancement and moderate amplitude to be crucial for good sectioning. For a Leica 1200S vibratome we used 0.2 mm/min blade advance speed and 0.5 mm amplitude. Methanol fixed heart tissue should use slower blade advancement, 0.14 mm/min.

Sections can be stored long term at 4° C. in PBS+0.1% Na-azide+1% Bovine serum albumin, or for longer periods can be frozen in 10% DMSO 90% PBS.

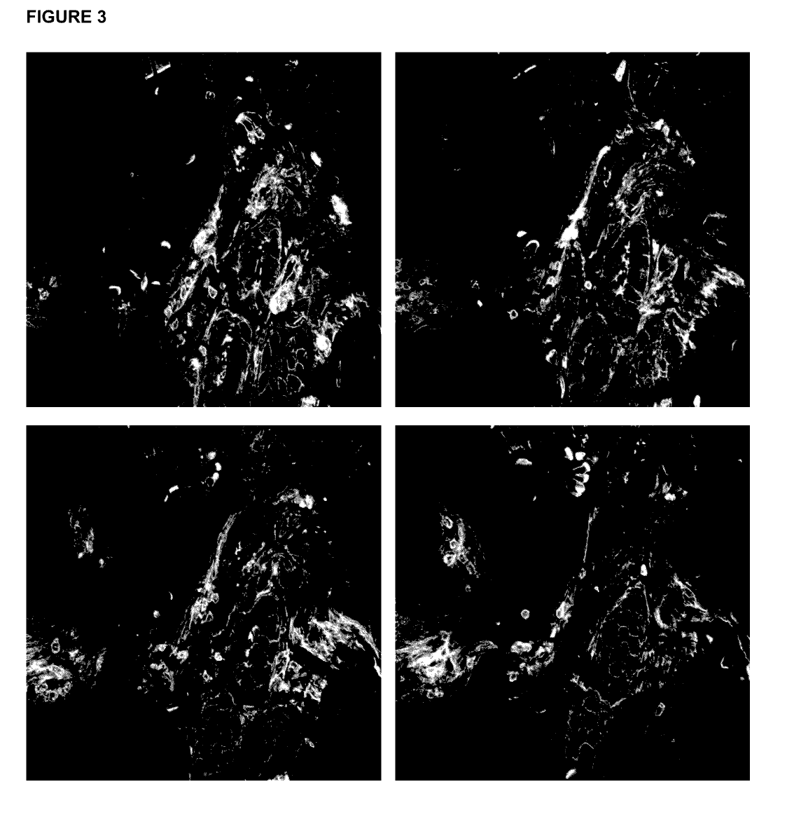

Intestine sample processed in this way resulted in sections with poorly preserved tissue morphology (FIG. 3). Though this method might be altered to work optimally in other tissues, it appears tissue specific optimization is required. Sectioning softer tissues, such as intestine, can be improved by utilizing more rigid embedding reagent (e.g,, higher % agarose).

### Example 6: Staining

We utilize a convenient staining protocol designed specifically for vibratome sections, which, in contrast to other sections, remain free-floating in suspension throughout the staining protocol, We use 24-well plates filled with buffer and custom-made baskets to hold the samples, The sample baskets are plastic cylinders that have a nylon mesh at one end, allowing the exchange of fluids from the sample within the basket and the buffers in the 24-well plate wells. These can be made with common lab items, such as Eppendorf tubes, cell strainers, and a hot plate. Other vessels may also be suitable, Incubation times may be varied for specific antibodies or stains.

Prepare 24-well plate with the solutions below in sequential order, use 600 uL of solution per well, transfer sample basket sequentially from well to well:

Wash/permeabilize×PBS+tritonX100 0.1% (PBST)×3

Block sample (e.g., 5% serum in PBST) for several hours

Incubate with primary antibodies at 4 C for 16-48 hours with rocking.

Wash PBST ×4

Incubate with secondary antibodies+chemical stains (such as DAPI, Hoechst, phalloidin, or wheat germ agglutinin, WGA) at 4 C for at 16-48 hours with rocking.

Wash PBST ×4

It is important to stain the samples in suspension, where reagents can flow through and make contact with all sides of the sample (in contrast to after being adhered to glass allowing contact from only one side), as this greatly improves the penetration of antibodies and dyes into the interior of the section,

### Example 7: Mounting and Clearing

Prepare glass slides by adhering two pieces of 300 μm thick aluminum foil tape (cut each piece to 2 mm×8 mm size) that are 7 mm apart. These will act as a bridge for the coverslip to lean on, preventing the sample from being crushed or squeezed.

Coat coverslip with adhesive media, we use 0.01% poly-L-lysine in PBS, let dry.

Transfer section onto the center of the coverslip with a brush, blot away residual PBS.

Place coverslip on a coverslip rack/caddy and submerge in the following solutions sequentially, 50 seconds each incubation.

70% isopropanol

85% isopropanol

95% isopropanol

100% isopropanol×2

Benzyl alcohol and benzyl benzoate at a 1:2 ratio (BABB)×3.

Keep sample in last BABB incubation. This step can be extended for thicker sections, though 10 minutes is sufficient for 100 μm sections. Electroporation may also be used to enhance clearing efficiency.

Place 80 uL of BABB onto the glass slide, between the two foil bridges and place the coverslip on the foil.

Seal with nail polish, epoxy, or other sealant.

Acquire confocal images with desired resolution (we used as high as 0.2 μm in xy, 0.3 μm in yz and xz) and analyze. Unless noted images were obtained with 60× objective. The NIH's ImageJ is suitable free software for rendering 3D images and performing quantifications.

We have tested other mounting methods, such as mounting in Mowiol and ProLong Gold; sample processing without BABB clearing/mounting causes problems during image acquisition, where the section interior cannot be imaged due to poor light penetration. Other clearing reagents may also be suitable.

It is particularly useful to mount the sections on the coverslips, rather than on the slide. Conventional confocal microscopes have a narrow working distance when imaging with high resolution objective lenses (e.g., 60× objective), therefore any distance between coverslip and the sample will be subtracted from the total viewable thickness.

Samples can also be adhered to the coverslip by placing a small drop of molten low-melt agarose and spreading it around the sample, though the autofluorescence of agarose can result in non-specific signal in downstream imaging.

### Example 8: Observations

Thin sections with conventional methods

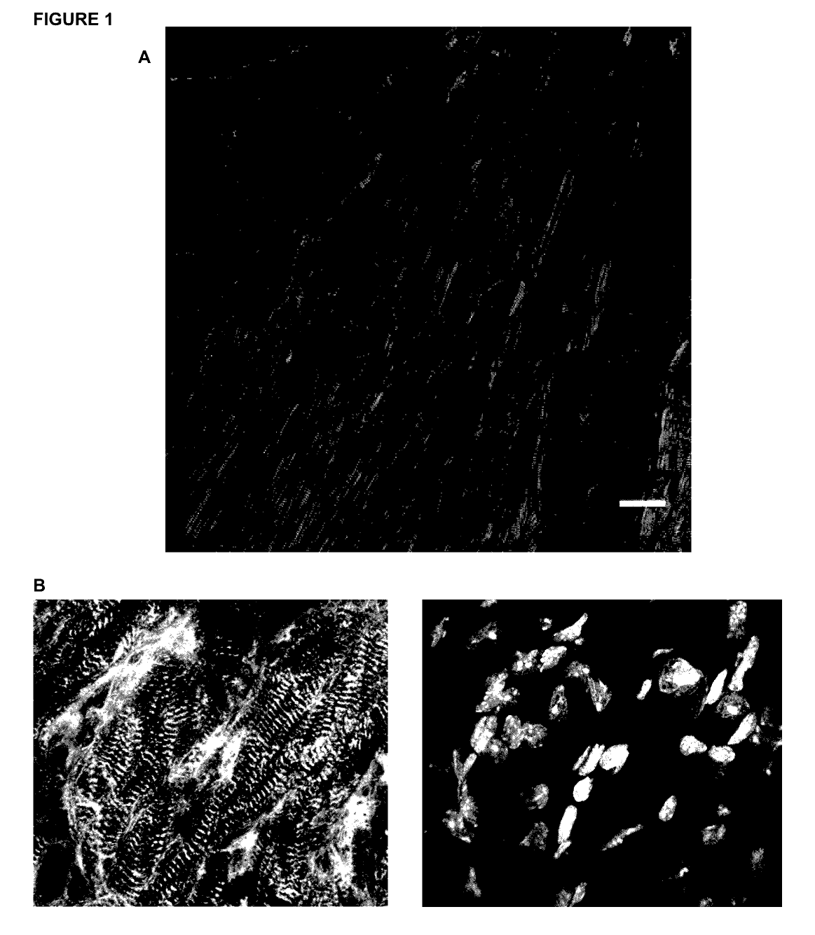

We found that, while the paraffin and cryosections of mice hearts were acceptable in many ways, the images suffered from their well-characterized limitations. Paraffin sections showed non-uniform signal when immunostained with alpha-actinin, a cardiac myocyte marker (FIG. 1A), likely due to damage to, or incomplete retrieval of antigens. Cryosections gave strong, uniform staining of cardiac myocyte sarcomeres, but the nuclear morphology was visibly distorted (FIG. 1B). Despite these drawbacks, thick sections generated from non-vibratome sectioning methods may be suitable/compatible with our staining/clearing/mounting protocols.

High Resolutio in 3D with Preserved Morphology

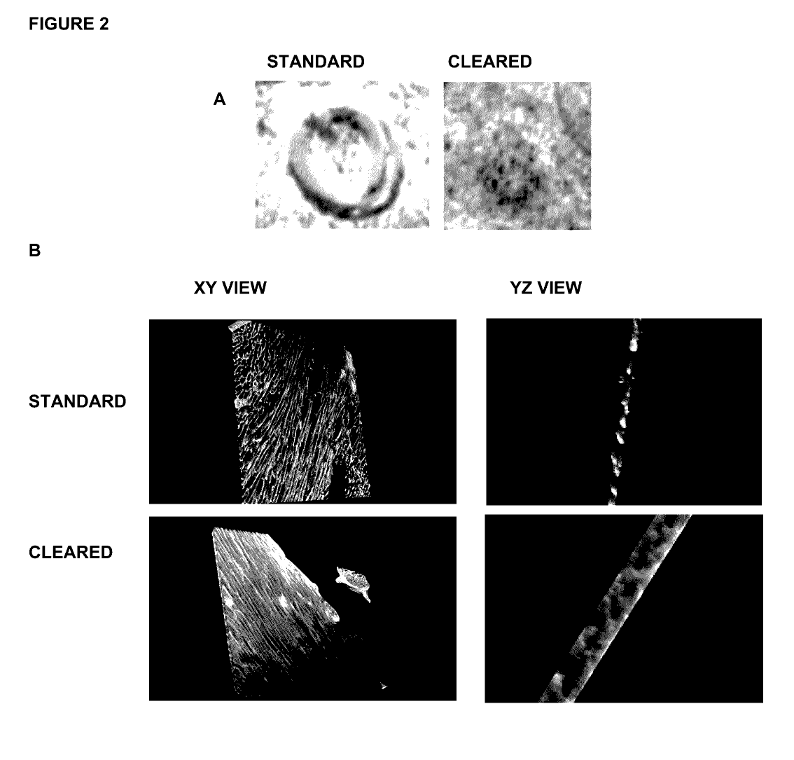

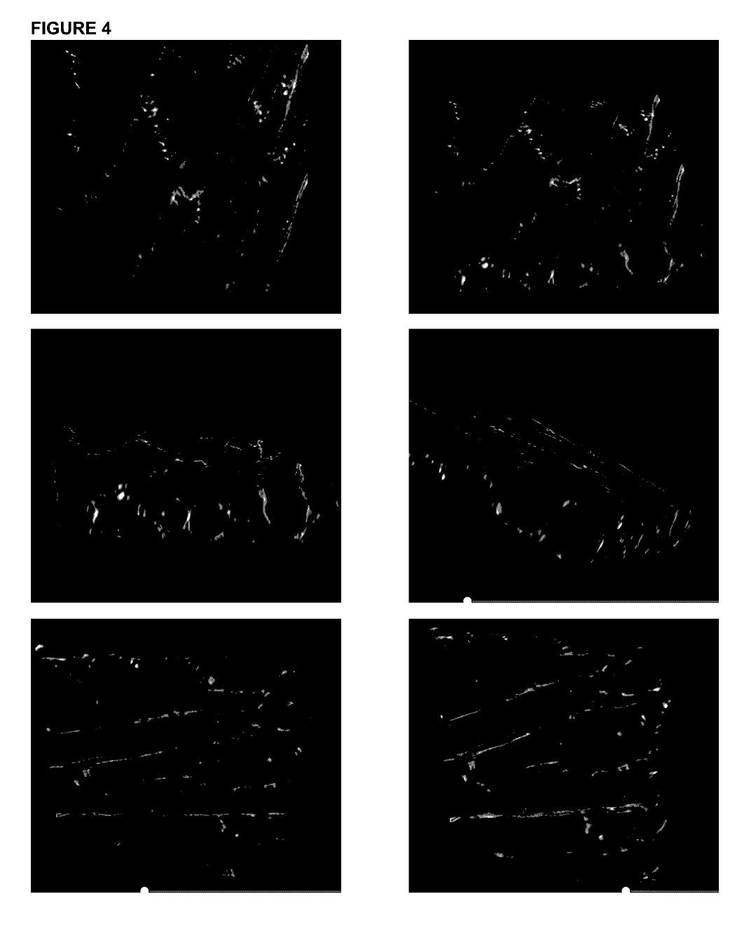

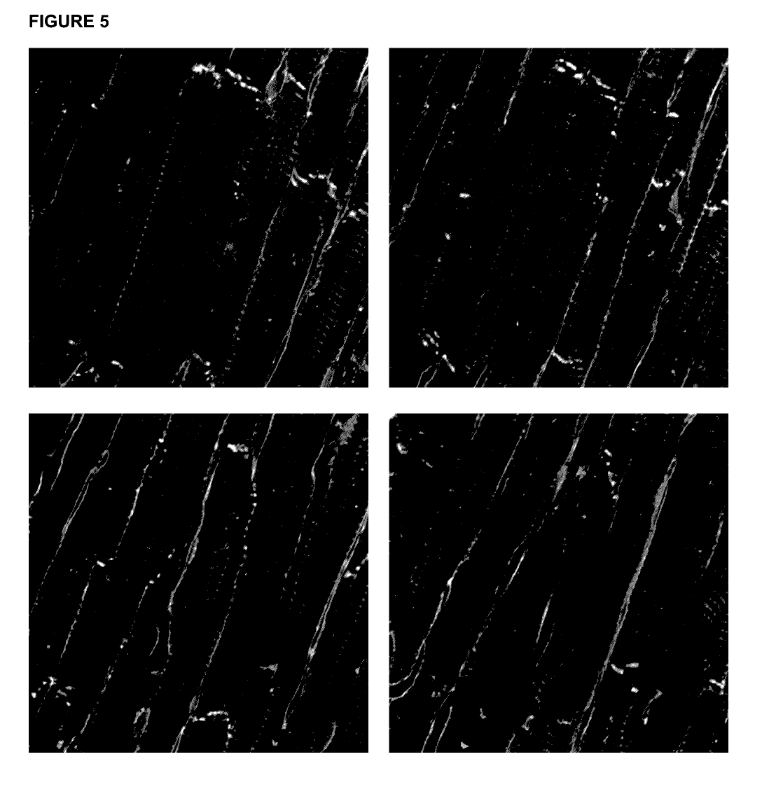

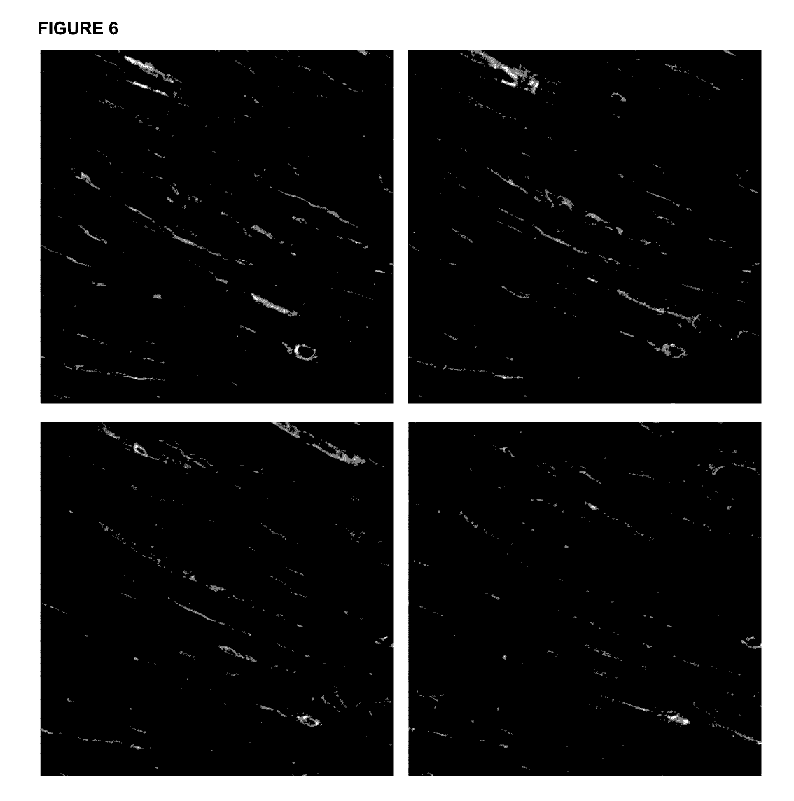

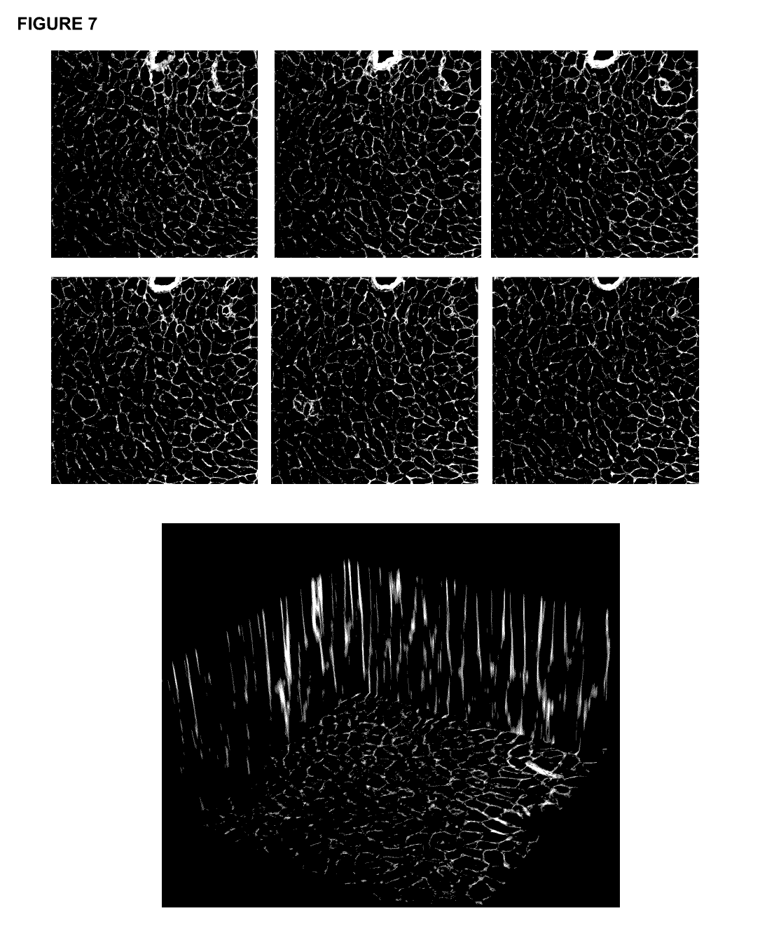

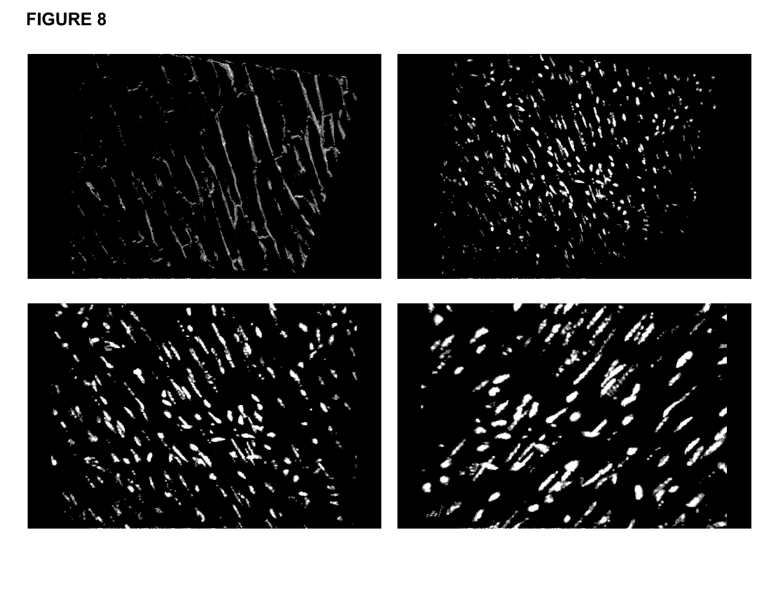

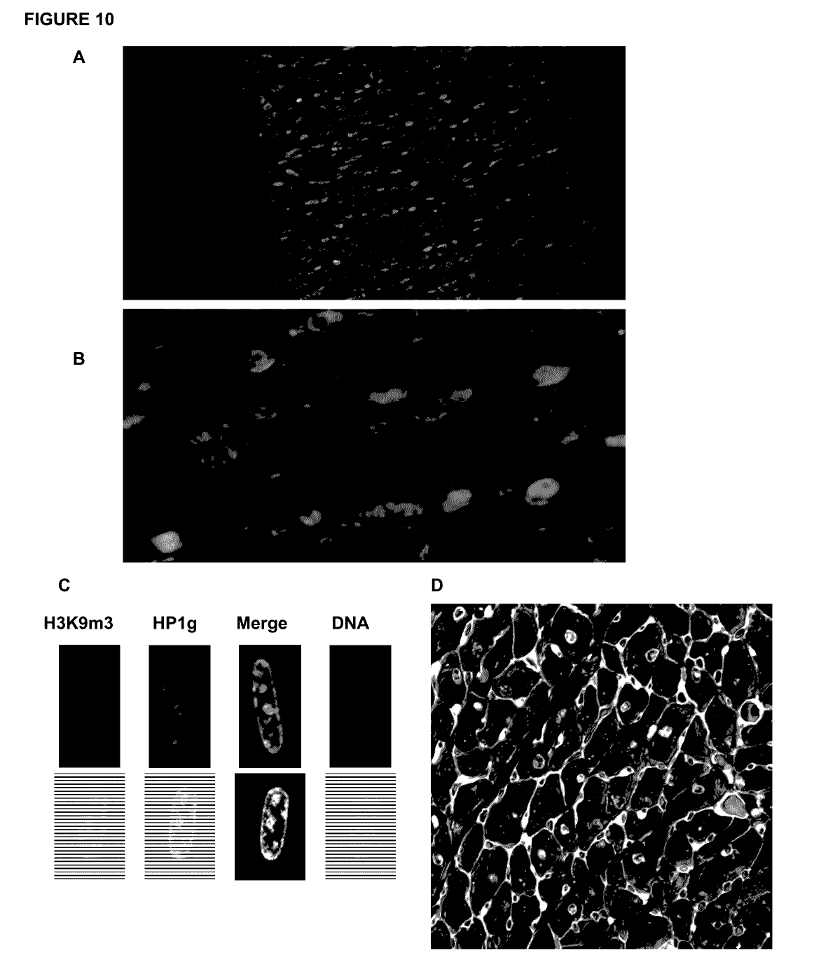

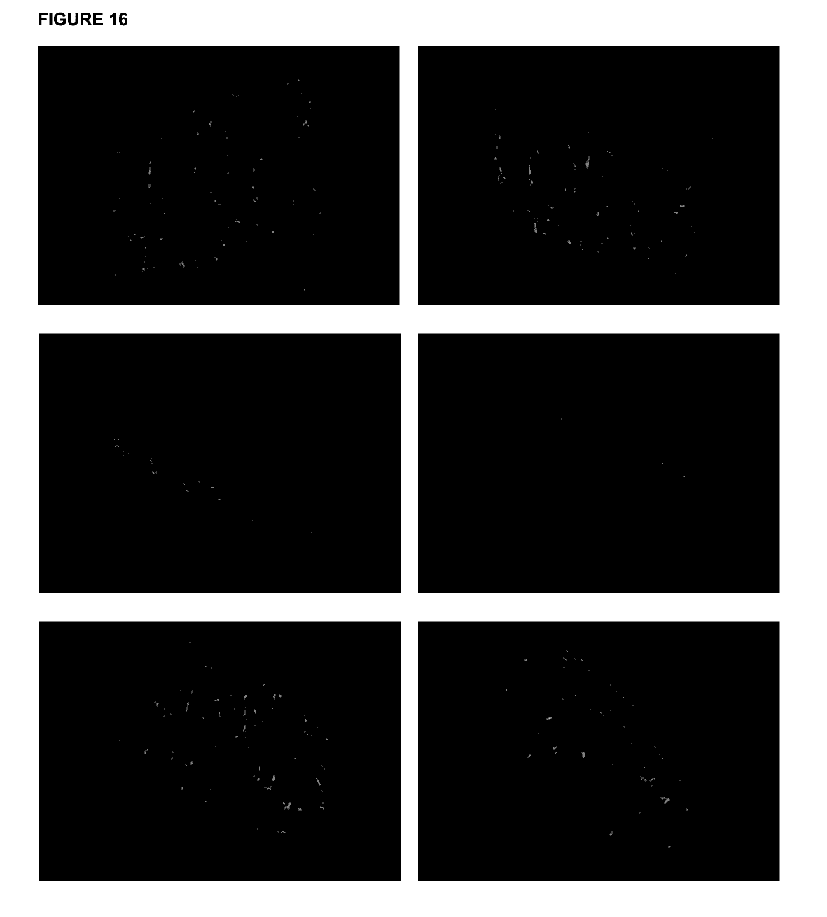

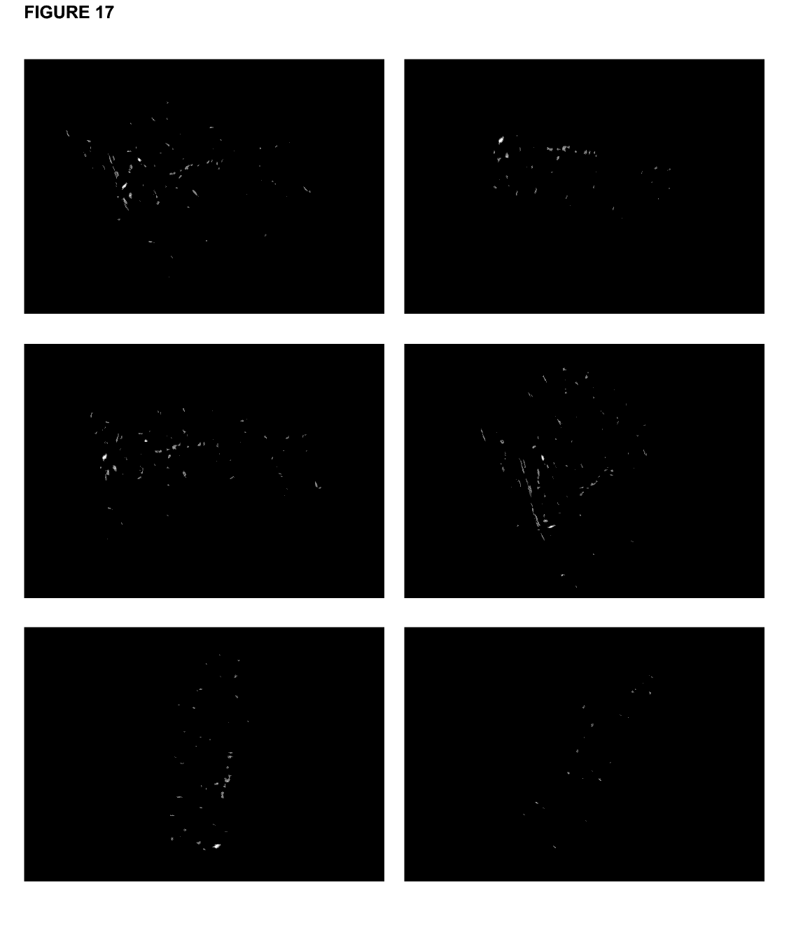

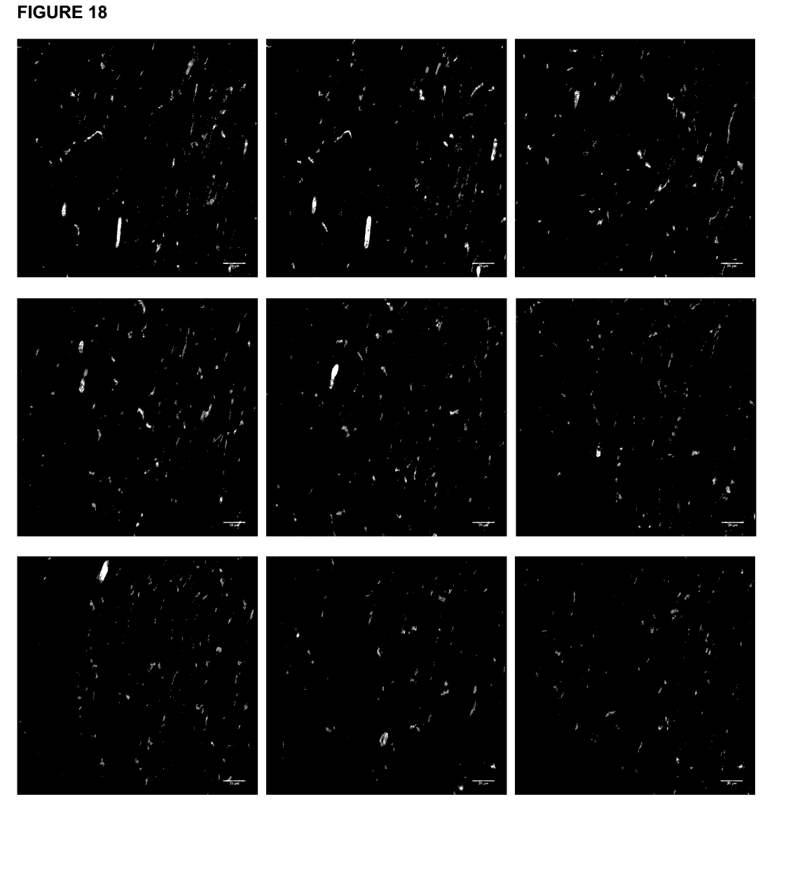

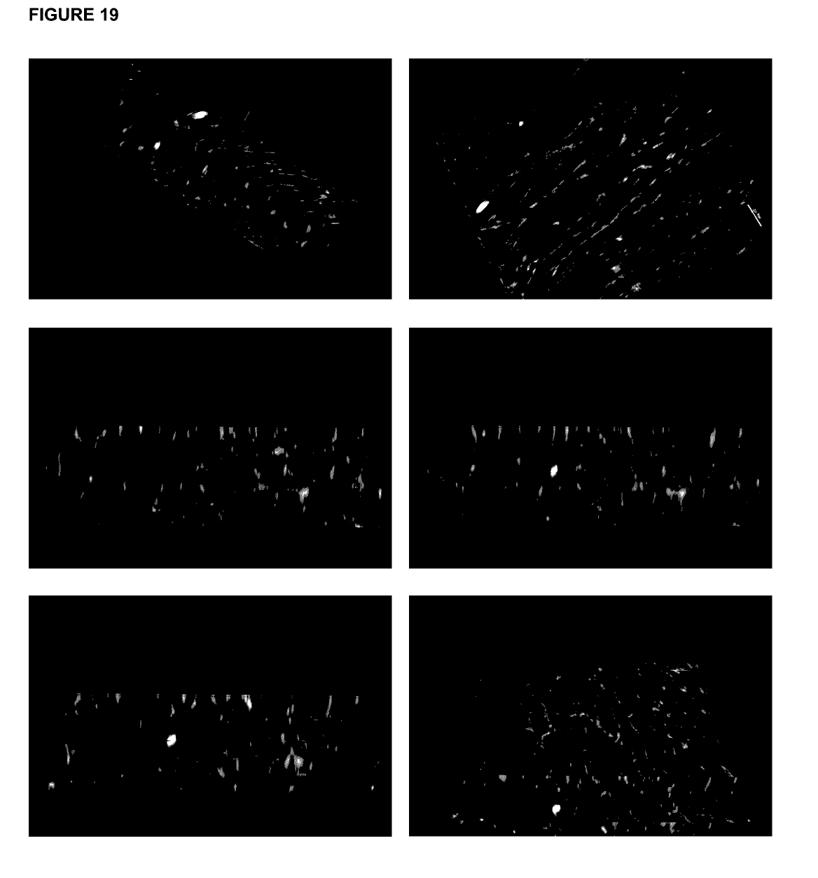

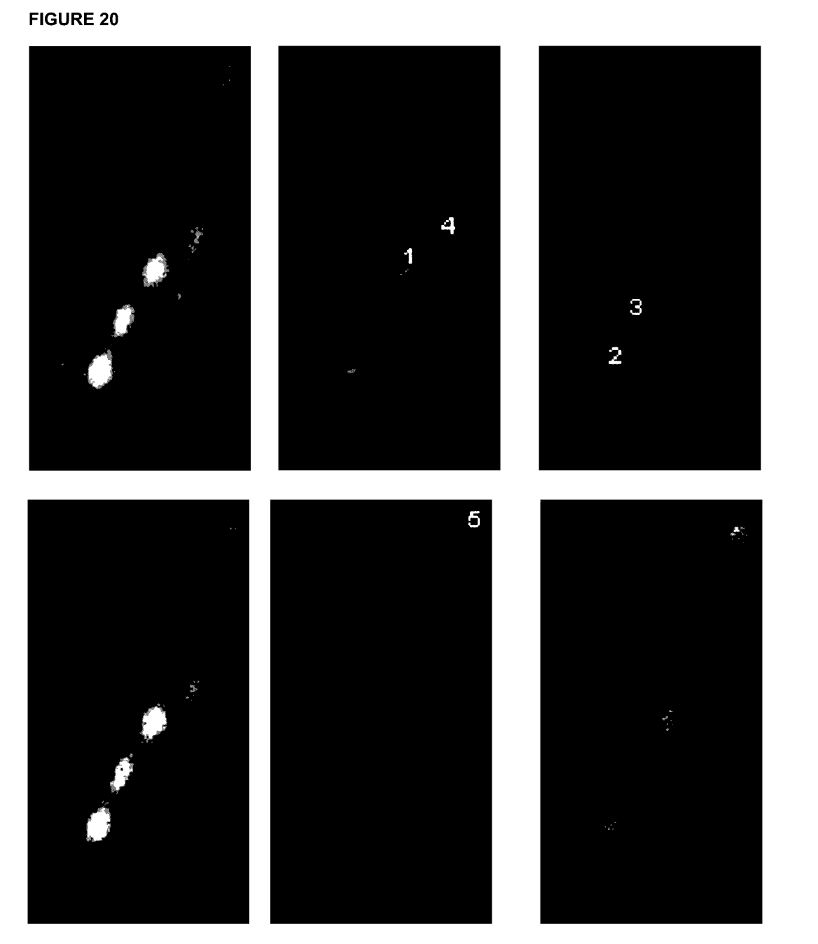

We stained the samples fixed with either PFA or MeOH and found both fixation methods were suitable for this approach (FIGS. 4-6). Clearing specimens with BABB was effective at making the samples transparent (FIG. 2A). This was critical for downstream imaging, as seen by the lack of signal after viewing a few microns into the depth of sections of standard non-cleared specimens (FIG. 2B), in contrast to the cleared samples showing signal throughout the depth. 3D images were obtained and showed uniform staining of sarcomeres and preserved morphology of cells and nuclei with clearing. Details of sub-micron sized objects, such as sarcomeres and DNA/chromatin structures within the nuclei were clearly visualized, while simultaneously, larger features, including gap junctions (WGA and phalloidin double positive), alignment of myocytes, large blood vessel and capillary networks, could be visualized through several layers deep of whole/intact cardiac myocytes in their native context (FIGS. 4-10 and 15-21).

Visualizing Fibrosis and Sarcomere Disarray in Human Ischemic Heart Disease

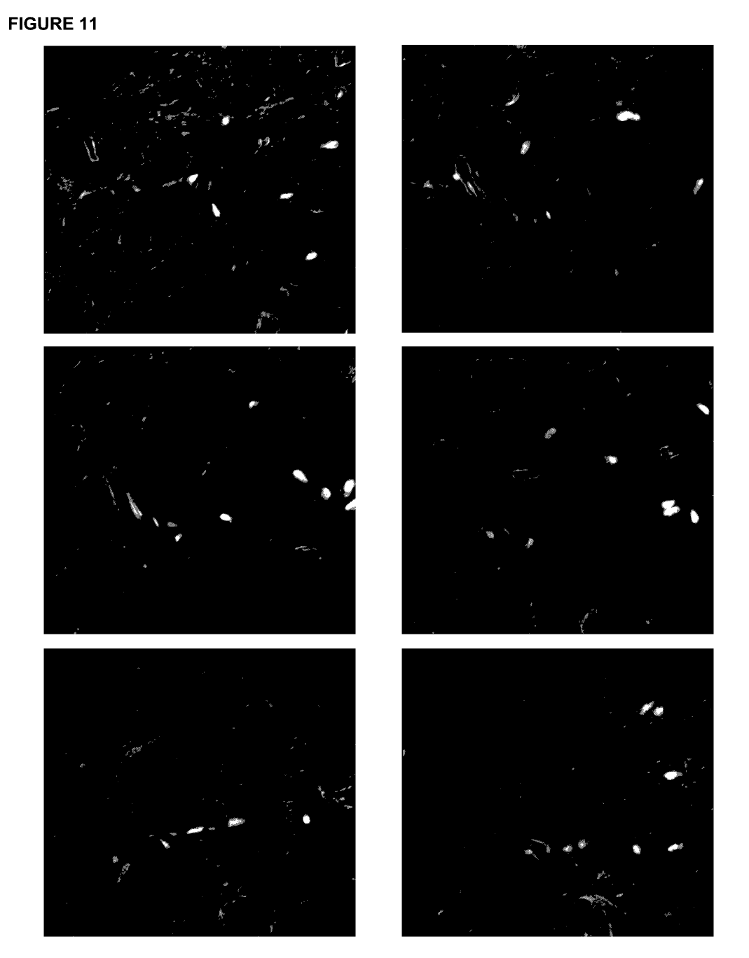

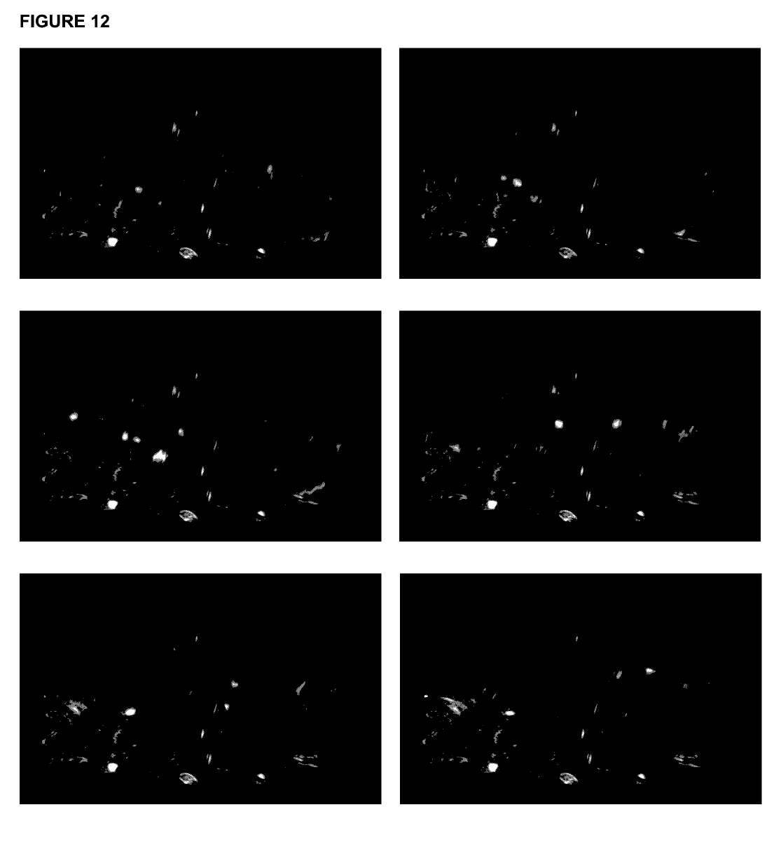

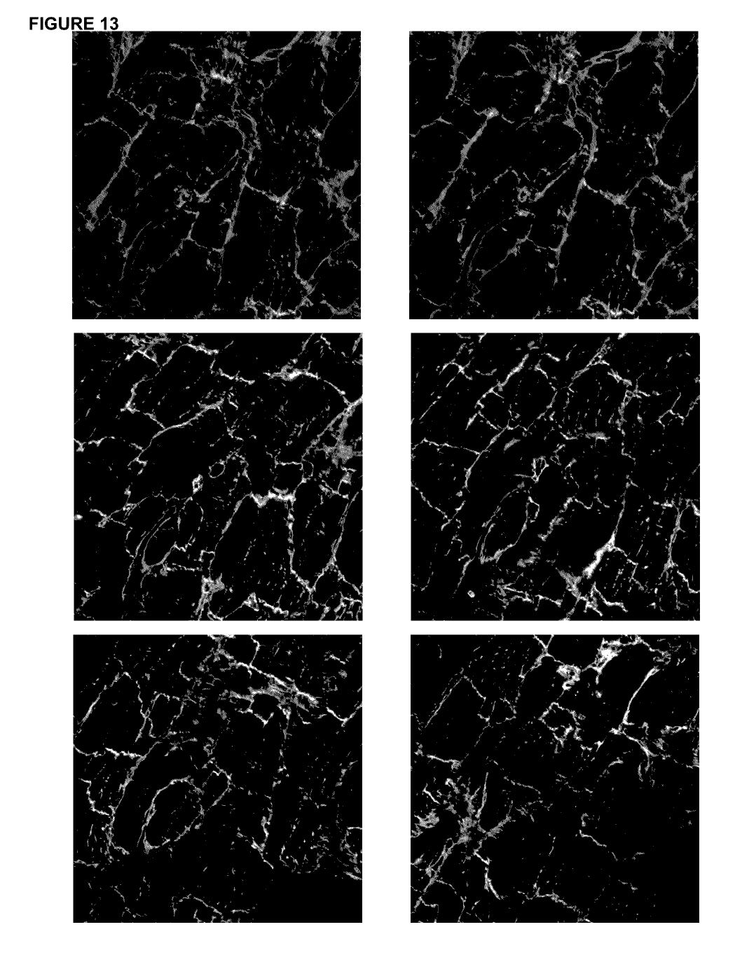

Staining with WGA, which is a fluorescently labeled lectin that binds glycoproteins rich in extracellular matrix, we observed marked accumulation of extracellular matrix in human ischemic heart disease samples (FIGS. 11-13), suggesting fibrosis had occurred. Though some regions of the heart contained myocytes with preserved sarcomere structure (FIGS. 11, 12), some regions clearly showed disorganization of the sarcomeres (FIG. 13).

Quantifying Rare Mitotic Cardiac Myocyte in Adult Mice

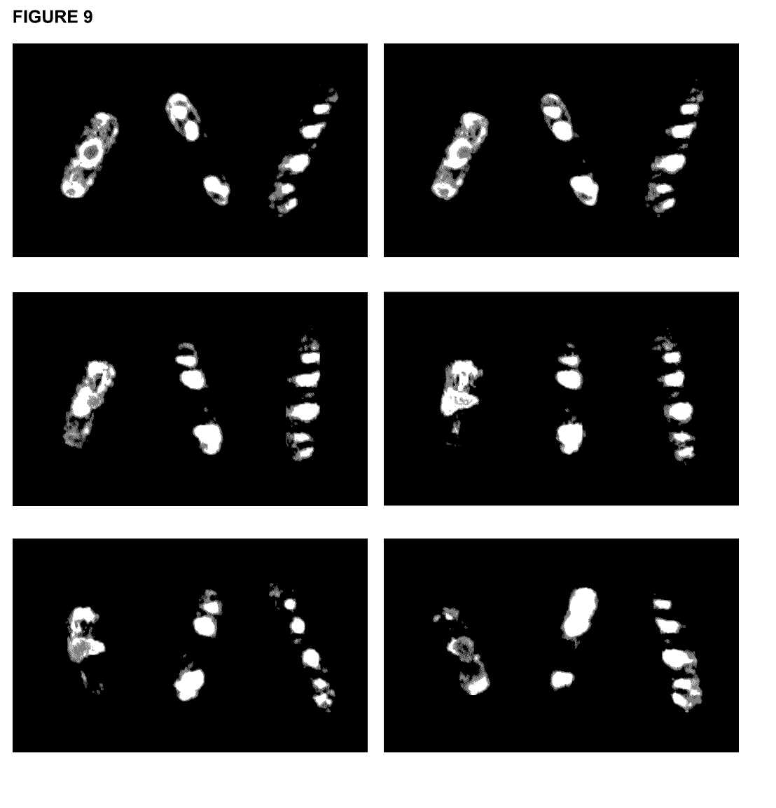

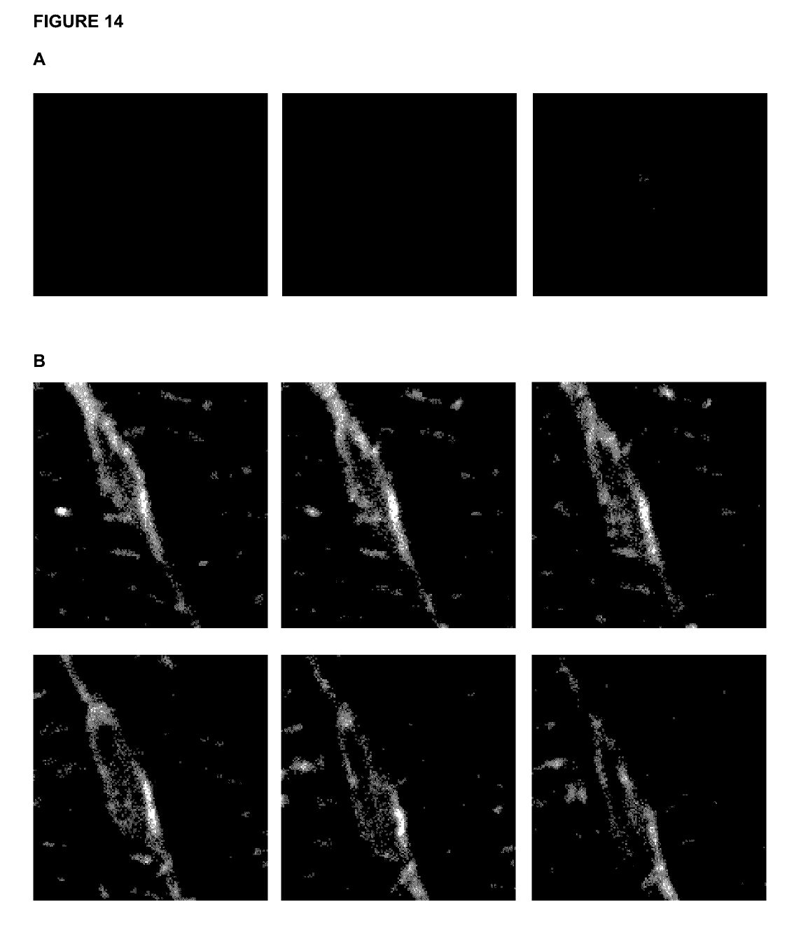

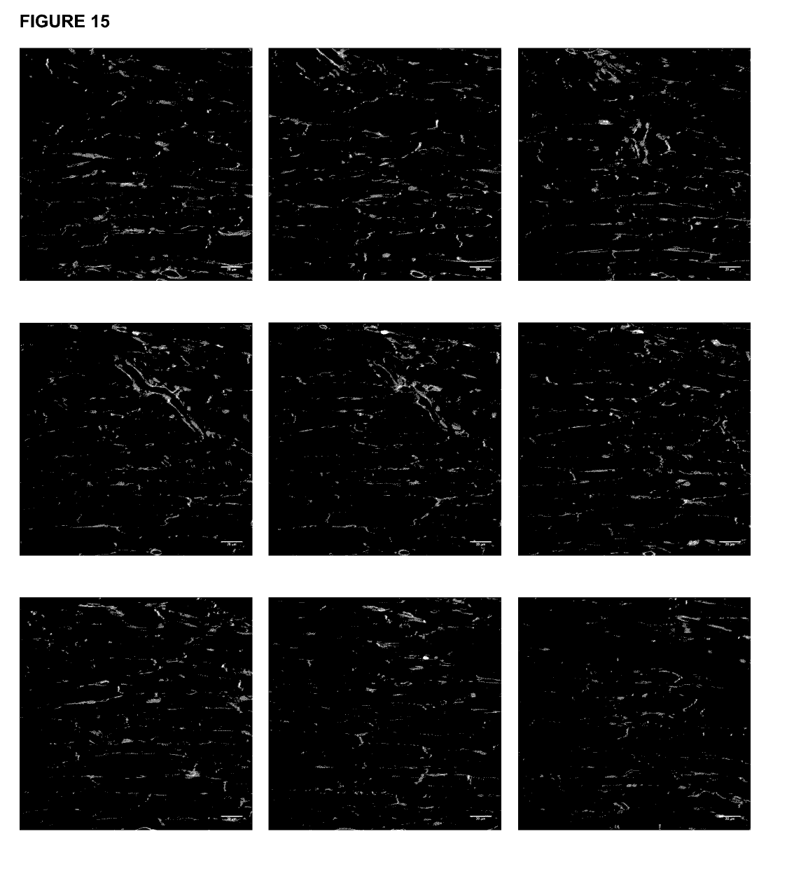

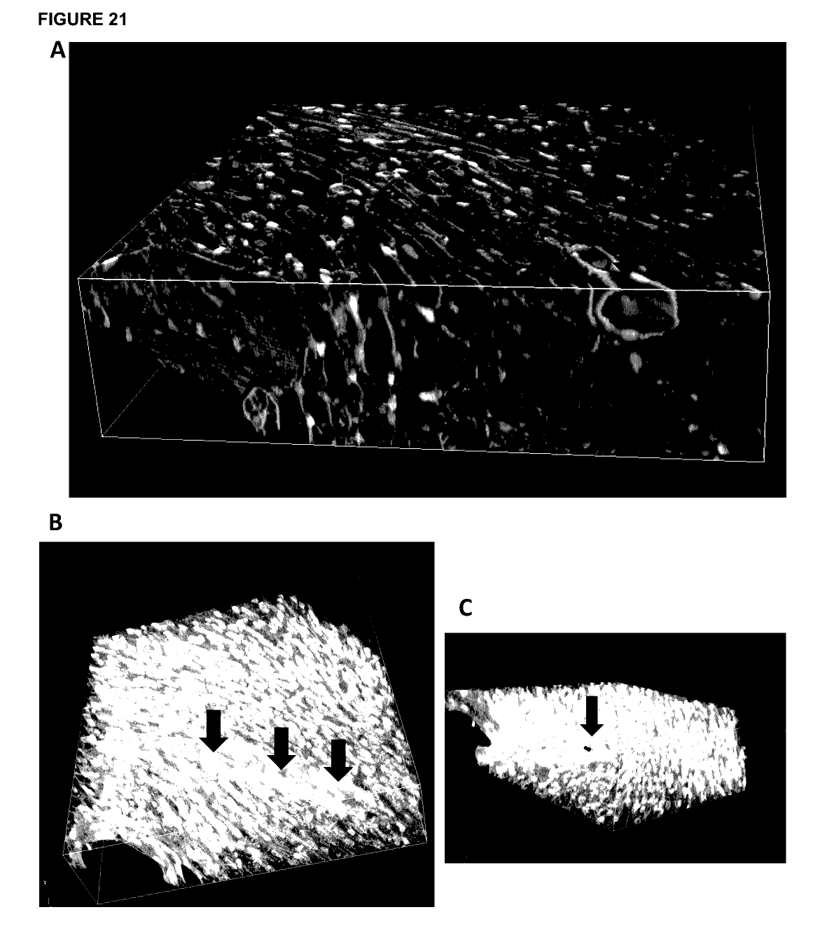

To quantify the mitotic activity of mammalian adult cardiac myocytes in situ, we stained vibratome sections for phosphorylated-Histone H3 (pH3), a marker of mitosis. We studied normal and genetically engineered mice that overexpress cell cycle-activating genes. After growth stimulation by trans-aortic constriction (TAC), we clearly observed pH3+ cardiac myocytes in the transgenic mice. We note that traditional methods may lead to false positives, as without 3D context, it can be difficult to accurately assign the nucleus of a cell as a cardiac myocyte or another cell type. For example, a 2D image (FIG. 14A) appears to Showa nucleus within sarcomere structures, suggesting it is a cardiac myocyte nucleus, however, upon 3D imaging (FIG. 14B), it is dear the nucleus is within an endothelial cell of a capillary. We were able to quantify the amount of mitotic cardiac myocyte nuclei within a given volume, the volumes of cardiac myocytes, and total myocardial volume, which allowed direct quantification of the amount of proliferating cardiac myocytes, in contrast to 2D measurements, which rely on many assumptions to indirectly estimate volume. As expected, the transgenic mice with activated cell cycle showed a dramatic increase of mitotic adult cardiac myocytes: 1,324 per control heart vs. 82,422 per transgenic heart (FIGS. 15-19). We note that direct quantitative volume measurement was possible for a wide range of structures, ranging from chromatin domains in nuclei (<1 μm³) (FIG. 9, 20) to whole adult cardiac myocytes (>60,000 μm³), and our strategy is amenable to high resolution (0.3 μm or better) 3D quantifications in intact cardiac cells in their native context for volumes of greater than 1×10⁸ μm³ (FIG. 21), which can be further increased by utilizing image “stitching” techniques and thicker sections.

### Discussion

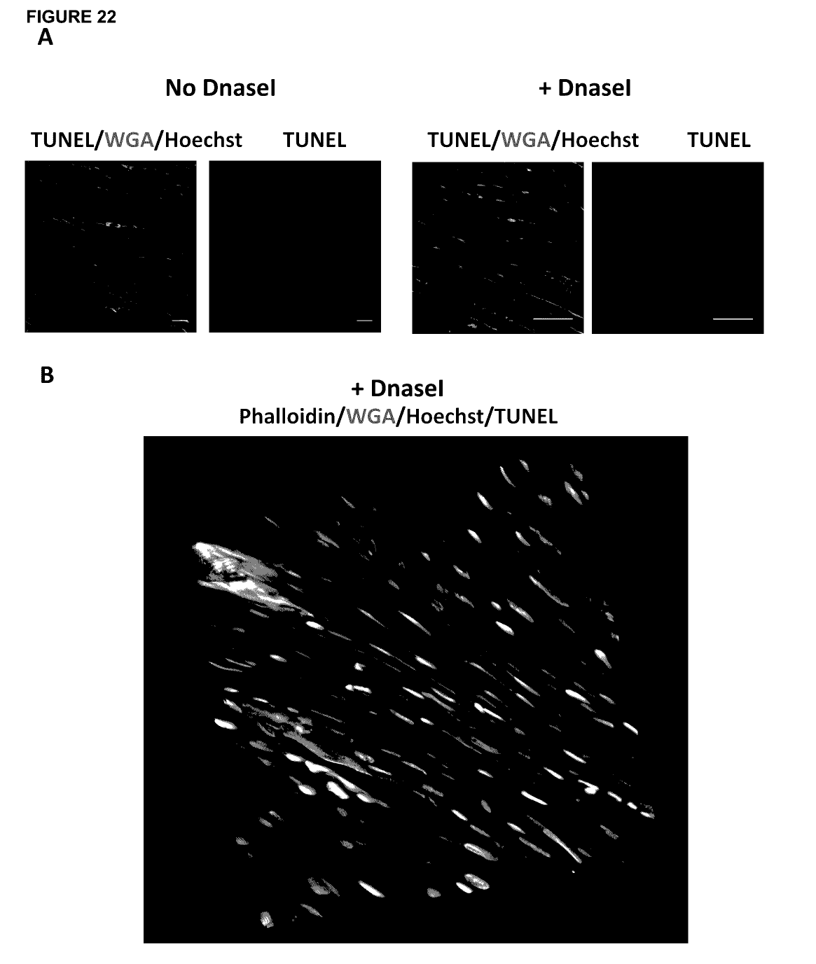

In the most recent decades, light microscopy has progressed tremendously, with resolution levels going well below the diffraction limit of light. Sample preparation, in stark contrast, has remained relatively stagnant, with the most common practices today very closely resembling that of 50 years ago. We have described a novel method for quantitative high resolution 3D imaging in mammalian hearts that provides information of whole cardiac cells within native tissue. We showed that our sample preparation and staining method can be used for direct 3D quantitative and morphological studies of: cardiac cells, myocyte alignment, nuclei, chromatin, vascular networks, fibrosis, sarcomeres alignment, cell cycle activity, and other assays including apoptosis/cell viability assays (Click-iT detection) (FIG. 22). Importantly we show that chemical dyes (DAPI, Hoechst, phalloidin, and wheat-germ agglutinin/lectin-labeled probes, etc), antibody-based stainings, and Click-iT detection methods were all compatible with the 3D imaging and quantifications we have discussed. This innovation will highly impact other areas or cardiology, in basic, translation and clinical research areas. The fact that no other method has been able to produce comparable images in adult mammalian hearts, attests to the uniqueness and utility of this innovation, which is comprised of a 20-step protocol and reagents that have been specifically optimized to produce the desired results.

Stem cell-derived cardiac myocytes as a cell transplantation therapy is coming to fruition. There are valid concerns regarding the connectivity of graft tissue with the host, as studies in non-human primates indicate an increase of arrhythmias that accompanies the increased heart function after cell transplantation in a myocardial infarction model (8). Our innovation will empower future studies that assess cell therapy engraftment, allowing visualization of myocyte alignment, gap junctions, and vessel network between graft and host tissue. Other cardiac cell therapy systems, such as cardiosphere cells, would also gain from this innovation, as cardiospheres are frequently dissociated to allow single cell analysis, whereas the cells could be viewed in the context of the cardiosphere with this technology.

Obtaining myocardium biopsies in humans is very rarely indicated, despite major advancements in the field of cardiac catheterization. This is largely due to the fact there is little useful information that can be gained from these samples using traditional methods, as they are unable to capture intact, whole cardiac myocytes, which can be hundreds of times larger than other cardiac cells. The innovation described in this work will be useful to better understand the nuances of very broad disease classifications, such as ischemic heart disease. High-resolution, 3D Visualization of fibrosis, sarcomere disarray, myocyte organization/alignment, nuclear morphology, vasculature, and other features, will undoubtedly advance our understanding of heart disease, and may provide novel diagnostic tools.

### Example 9: Super-Resolution Microscopy

The sample preparation protocol described above has been used with super-resolution microscopy and found to work well with this imaging modality. The super-resolution data were acquired using a Leica iSIM-superresolution spinning disk confocal microscope. The use of the spinning disk also greatly decreased image acquisition time.

### REFERENCES

1. P. M. Treuting, S. M. Dintzis, Comparative Anatomy and Histology: A Mouse and Human Atlas (Expert Consult). (Academic Press, 2011).

2. S. Feldengut, K. Del Tredici, H. Braak, Paraffin sections of 70-100 μm: a novel technique and its benefits for studying the nervous system. J Neurosci Methods 215, 241-244 (2013).

3. E. Avolio et al., Combined intramyocardial delivery of human pericytes and cardiac stem cells additively improves the healing of mouse infarcted hearts through stimulation of vascular and muscular repair. Circ Res 116, e81-94 (2015).

4. A. M. Gerdes et al., Structural remodeling of cardiac myocytes in patients with ischemic cardiomyopathy. Circulation 86, 426-430 (1992).

5. M. Kaneko, S. Coppen, S. Fukushima, M. Yacoub, K. Suzuki. (J Cell SciTher, 2012), vol. 3:126.

6. C. E. Miller et al., Confocal imaging of the embryonic heart: how deep? Microsc Microanal 11, 216-223 (2005).

7. T. Yokomizo, T. Yamada-Inagawa, A. Yzaguirre, M. Chen, N. Speck, E. Dzierak. Whole-mount three-dimensional imaging of interally localized immunostained cells with mouse embryos. Nature Protocols. Vol 7. No3. 421-431. 2012.

8. J. J. Chong et al., Human embryonic-stem-cell-derived cardiomyocytes regenerate non-human primate hearts. Nature 510, 273-277 (2014).

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A method of preparing a tissue sample for high-resolution three-dimensional tissue imaging, comprising:
(a) embedding a fixed tissue sample in an embedding medium;
(b) sectioning the embedded tissue into sections of 20-1000 μm thickness;
(c) staining the sectioned tissue;
(d) mounting the stained 20-1000 μm thick tissue sections on a coverslip and glass slide; and
(e) clearing the 20-1000 μm thick tissue sections with a reagent that solubilizes lipids, increases sample transparency, and increases sample refractive index.

2. The method of claim 1, wherein the tissue is adult mammalian tissue.

3. The method of claim 1, wherein the tissue is cardiac tissue.

4. The method of claim 1, wherein the tissue is intestinal tissue.

5. The method of claim 1, wherein the tissue sections are 50-500 μm thick.

6. The method of claim 1, wherein the clearing reagent of step (e) is a mixture of Benzyl Alcohol and Benzyl Benzoate (BABB).

7. The method of claim 1, wherein the tissue is fixed with formaldehyde, paraformaldehyde, glutaraldehyde, acetone, methanol, ethanol, or isopropanol.

8. The method of claim 7, wherein the tissue is fixed in 4% paraformaldehyde in PBS, precooled to 4° C., and incubated about 16 hours at 4° C.

9. The method of claim 7, wherein the tissue is fixed in 100% methanol (MeOH), precooled to −20°, incubated at about −20° C. for 30 min to 1 hr; and in a stepwise manner, incubated in precooled MeOH at about 80% MeOH/20% PBS, then about 60% MeOH/40% PBS, wherein each incubation is about 30 minutes at about −20° C.

10. The method of claim 1, wherein the embedding medium is agarose, histogel, paraffin, or optimal cutting temperature (OCT) compound.

11. The method of claim 1, wherein the staining comprises incubating the sectioned tissue in suspension with fluorescent, luminescent, and/or staining reagents.

12. A method of high-resolution three-dimensional tissue imaging, comprising preparing a tissue sample according to the method of claim 1, and acquiring images of the tissue with desired resolution.

13. The method of claim 12, wherein the desired resolution is about 0.2 μm in xy, and about 0.3 μm in yz and xz.

14. The method of claim 12, wherein the images are obtained with a 60× or 100× objective.

15. The method of claim 12, wherein the acquisition of images comprises confocal, spinning disc, light sheet, stochastic optical reconstruction microscopy (STORM), photoactivated localization microscopy (PALM), stimulated emission depleted (STED), NSOM, or SOFI imaging.

16. The method of claim 1, wherein the staining comprises use of a fluorescent or luminescent protein.

17. A kit comprising a plurality of pre-coated adherent ultra-thin-coverslips (#0), BABB, embedding medium, a plurality of baskets comprising 100 μm or smaller mesh for in-suspension staining in wells, and at least one container.

18. The kit of claim 16, further comprising a package insert containing instructions for use.
